(19) United States
(12) Patent Application Publication
(10) Pub. No.: US 2026/0315702 A1
(43) Pub. Date: Oct. 15, 2026

(54) Medicament Delivery System with Dose Divider

(71) Applicant: SANOFI PASTEUR, INC., Swiftwater, PA (US)

(72) Inventor: Stephan Riedel, Frankfurt am Main (DE)

(21) Appl. No.: 19/482,155

(22) PCT Filed: May 10, 2024
(86) PCT No.: PCT/US2024/028807

§ 371 Date: Nov. 6, 2025

(30) Foreign Application Priority Data
May 11, 2023 (EP) 23172953.4

(51) Int. Cl. A61M 5/315

(52) U.S. Cl. CPC A61M 5/31595

(57) ABSTRACT

A medicament delivery system comprises a syringe, a plunger rod is, and a guide element at a proximal end of the syringe and comprising an aperture through which the plunger rod extends. The guide element and the plunger rod are relatively rotatable between blocking and release positions. A guide mechanism provided on the plunger rod and the guide element permits the plunger rod to move axially over a first range of motion relative to the syringe from an initial position to an intermediate position to expel a first dose of medicament, and blocks the plunger rod from moving over a subsequent range of motion relative to the syringe. The plunger rod is free to move in an axial direction over the subsequent range of motion to expel further medicament upon relative rotation of the guide element and the plunger rod from the blocking position to the release position.

### TECHNICAL FIELD

The present disclosure relates to a medicament delivery system configured to facilitate delivery of a medicament in separate doses.

### BACKGROUND

Medicament delivery devices may typically comprise a container of medicament having an outlet through which the medicament can be expelled from the container during medicament delivery, and a plunger rod operable by a user to cause the delivery of medicament from the outlet. Such plunger rod may, for example, comprise a plunger or piston within the medicament container which can be moved into the medicament container to dispense medicament.

Some medicaments are intended to be delivered in two or more doses from a single container. In such cases, a user may be required to manipulate the plunger rod over a first range of motion to dispense a first dose, and then manipulate the plunger rod over one or more subsequent range(s) of motion to dispense subsequent doses. Some patients may have difficulty in manipulating the plunger rod to accurately dispense the intended separate doses with the intended quantities of medicament in each dose. Alternatively, patients may accidentally dispense unintended quantities of medicament, or dispense all of the medicament at once, through inexperience with the device or misunderstanding of device usage instructions.

A known device for delivering divided doses of medicament is available as FluMist™ device. Other known devices for delivering divided doses of medicament are disclosed in WO 2022/049080 and WO 2022/049086.

Respiratory syncytial virus (RSV) is an important cause of severe acute lower respiratory illness (LRI) in infants and children and a common cause of severe pneumonia requiring hospital admission in children worldwide. According to global estimates, RSV caused approximately 33 million cases of LRI and approximately 118,000 deaths in children <5 years of age in 2015. Greater than 80% of all RSV-associated LRIs (RSV-LRIs) and more than 50% of the RSV-associated deaths in low- and middle-income countries were estimated to occur in infants ≥6 months old.

### SUMMARY

According to the present disclosure, there is provided a medicament delivery system comprising a syringe comprising a chamber for containing medicament and an outlet at a distal end of the syringe through which medicament can be expelled from the chamber, a plunger rod having a longitudinal axis, mounted within the syringe and slidable relative to the syringe in an axial direction of the plunger rod to cause medicament to be expelled from the chamber when medicament is within the chamber, a guide element at a proximal end of the syringe and comprising a central aperture through which the plunger rod extends, wherein the guide element and the plunger rod are relatively rotatable about the longitudinal axis of the plunger rod between a blocking position and a release position, and a cooperating guide mechanism provided on the plunger rod and the guide element, wherein the guide mechanism permits the plunger rod to move in an axial direction over a first range of motion relative to the syringe from an initial position to an intermediate position to expel a first dose of medicament, and blocks the plunger rod from moving over a subsequent range of motion relative to the syringe to expel further medicament, and wherein the plunger rod is free to be moved in an axial direction over the subsequent range of motion relative to the syringe to expel further medicament upon relative rotation of the guide element and the plunger rod from the blocking position to the release position.

The guide element may comprise a sleeve or collar. The guide element may be substantially cylindrical or tubular.

The guide element may be in a start position relative to the plunger rod when the plunger rod is in the initial position, the guide element may be in the blocking position relative to the plunger rod when the plunger rod is in the intermediate position, and the guide element may be in the release position relative to the plunger rod when the plunger rod is free to be moved over the subsequent range of motion to expel further medicament.

The start position and the release position of the guide element may be the same.

The start position and the intermediate position of the guide element may be the same.

The start position and the intermediate position of the guide element may be rotationally offset about the axis of the plunger rod.

The guide mechanism may be configured to cause relative rotation between the guide element and the plunger rod as the plunger rod moves from the initial position to the intermediate position.

The guide mechanism may comprise a follower or pin formed on one of the guide element and the plunger rod, and a track within which the follower or pin is received, formed on the other of the guide element and the plunger rod.

The guide track may comprise a first section, a second section, and a transition section connecting the first and second sections.

The guide track may comprise a first section along which the follower travels when the plunger rod moves over the first range of motion, a second section along which the follower travels when the plunger rod moves over the subsequent range of motion, and a transition section connecting the first and second sections, and wherein the plunger rod is prevented from moving over the second range of motion until the follower has travelled along the transition section from the first section to the second section.

The first section may be disposed at angle α relative to the longitudinal axis of the plunger rod, wherein the 0°<α<90°.

The first section may be disposed substantially parallel to the longitudinal axis of the plunger rod.

The second section may be substantially parallel to the longitudinal axis of the plunger rod.

The transition section may be substantially perpendicular to the longitudinal axis of the plunger rod.

The guide element may be connected to the syringe by a biasing member configured to rotationally bias the guide element relative to the plunger rod from the blocking position to the release position. The biasing member may comprise a ring-shaped member and may comprise an elastomeric ring, and may include a central aperture. The biasing member may comprise a coil spring or a torsion spring.

The biasing member may be resiliently deformable as the plunger rod moves from the initial position to the intermediate position.

The biasing member may be resiliently deformed when the plunger rod is in the initial position. The biasing member may be in a relaxed or substantially undeformed state when the plunger rod is in the initial position.

The guide element may be free to move under the action of the biasing member to the release position when the follower enters the transition section of the guide track.

The syringe may comprise a component at, attached to, or configured to attach to, a proximal end of the syringe. The component of the syringe may be a separate component, or may be a part integrally formed with the syringe. The syringe may comprise a lip at a proximal end thereof, and the component may be configured to attach to the lip of the syringe. The component provided at or attached to the proximal end of the syringe may comprise a finger flange. The finger flange may be releaseably or detachably attached to the syringe. This may enable the guide element and/or finger flange to be fitted to known configurations of syringe, and/or retro-fitted to such syringes. The guide element may be coupled to the finger flange.

The biasing member may be secured to the finger flange.

The guide element and the plunger rod may be manually moveable between the blocking position and the release position.

The syringe may comprise a barrel and an outlet at a distal end of the barrel.

The syringe may comprise a piston in sealing contact with an inside wall of the barrel and slidable within the barrel by movement of the plunger rod to expel medicament from the outlet.

The plunger rod and guide element may be configured such that substantially half of the medicament in the chamber is expelled as the plunger rod moves over the first range of motion, and substantially half of the medicament in the chamber is expelled as the plunger rod moves over the subsequent range of motion.

The medicament delivery system may further comprise an atomiser at the outlet of the syringe. Such atomiser may facilitate insertion of the atomiser and outlet into an orifice of a patient into which the medicament is to be delivered. The atomiser and/or outlet may comprise a luer lock to sealingly connect the atomiser to the outlet. The medicament delivery system may comprise atomiser, plug, adaptor or other component at the outlet of the syringe. Such component at the outlet may comprise an atomiser, or may comprise a needle. A needle may be provided if medicament is intended to be delivered in doses into a patient in a particular manner. Such delivery may include intravenous delivery, subcutaneous delivery, and/or intramuscular delivery. The needle diameter may vary within the scope of the present disclosure, and may be selected according to the desired form of medicament delivery.

An atomiser, plug, adaptor or other component may be configured to atomise a liquid medicament as the medicament is expelled through the outlet and through a medicament passage in the atomiser, plug, adaptor or other component.

The system may be configured to deliver more than two doses of medicament. The system may be configured to divide the medicament into more than two doses, and may be configured to divide the medicament delivery into three, four, five, six or more doses. The system may comprise a plurality of guide track sections connected by transition sections, to enable multiple doses of medicament to be delivered.

Medicament may be received within the chamber of the syringe.

The medicament may any medicament described herein. The medicament may be a vaccine adapted for nasal administration. The vaccine may be an RSV vaccine.

The system may comprise an intranasal atomization delivery device.

The system may be configured to deliver about ½ of a dose to each nostril.

The system may be configured to deliver about 0.2 mL, wherein about 0.1 mL is delivered to each nostril.

The intranasal atomization delivery system may deliver an average droplet size Dᵥ₅₀ of about 10-120 μm.

The intranasal atomization delivery system may deliver an average droplet size Dᵥ₅₀ delivered to each nostril may be about 10-120 μm, about 30-110 μm, about 50-110 μm, about 70-110 μm, or about 80-110 μm.

The intranasal atomization delivery system may be configured so that an average shot volume to each nostril is between about 85 μL to about 120 μL, about 90 μL to about 115 μL, or about 95 μL to about 115 μL.

Also provided is an apparatus comprising a finger flange, guide element and a plunger rod for use with a syringe, the plunger rod receivable within an aperture in the guide element, wherein the guide element and plunger rod are relatively rotatable about the longitudinal axis of the plunger rod between a blocking position and a release position, and a cooperating guide mechanism is provided on the plunger rod and the guide element, wherein the apparatus is attachable to the syringe such that the plunger rod is slidable within and relative to the syringe, and such that the guide mechanism permits the plunger rod to move over a first range of motion relative to the syringe to expel a first dose of medicament, and blocks the plunger rod from moving over a subsequent range of motion relative to the syringe to expel a further dose of medicament.

The guide mechanism may enable the guide element and plunger rod to relatively rotate from the blocking position to the release position to permit the plunger rod to be free to be moved over a subsequent range of motion relative to the syringe to expel further medicament.

Also provided is a system comprising a finger flange, guide element and a plunger rod for use with a syringe, the plunger rod receivable within an aperture in the guide element, wherein the guide element and plunger rod are relatively rotatable about the longitudinal axis of the plunger rod between a blocking position and a release position, and a cooperating guide mechanism is provided on the plunger rod and the guide element, wherein the system is attachable to the syringe such that the plunger rod is slidable within and relative to the syringe, and such that the guide mechanism permits the plunger rod to move over a first range of motion relative to the syringe to expel a first dose of medicament, and blocks the plunger rod from moving over a subsequent range of motion relative to the syringe to expel a further dose of medicament.

In some embodiments, the finger flange is attachable to the syringe.

Also provided is a method of operating a medicament delivery system comprising a syringe comprising a chamber for containing medicament and an outlet at a distal end of the syringe, a plunger rod having a longitudinal axis, mounted within the syringe and slidable relative to the syringe to cause medicament to be expelled from the chamber when medicament is within the chamber, a guide element at a proximal end of the syringe and comprising an aperture through which the plunger rod extends, and a cooperating guide mechanism provided on the plunger rod and the guide element, the method comprising moving the plunger rod over a first range of motion relative to the syringe from an initial position to an intermediate position to expel a first dose of medicament, the guide element blocking the plunger rod from moving over a subsequent range of motion relative to the syringe, relatively rotating the guide element and the plunger rod from a blocking position to a release position, and moving the plunger rod over the subsequent range of motion relative to the syringe to expel a further dose of medicament.

The method may comprise initially inserting the outlet into a first nostril of a patient. The method may comprise inserting the outlet into a second nostril of a patient after relatively rotating the guide element and the plunger rod from a blocking position to a release position and before moving the plunger rod over the subsequent range of motion relative to the syringe to expel the further dose of medicament.

The method may comprise the guide element being in a start position relative to the plunger rod when the plunger rod is in the initial position, the guide element being in the blocking position relative to the plunger rod when the plunger rod is in the intermediate position, and the guide element being in the release position relative to the plunger rod when the plunger rod is free to be moved over the subsequent range of motion to expel further medicament.

The method may comprise the guide mechanism causing relative rotation between the guide element and the plunger rod as the plunger rod moves from the initial position to the intermediate position.

The method may comprise the follower moving along a first section of the guide track when the plunger rod moves over the first range of motion, the follower moving along a second section of the guide track when the plunger rod moves over the subsequent range of motion. The plunger rod may be prevented from moving over the second range of motion until the follower has travelled along a transition section of the guide track from the first section to the second section.

The method may comprise the guide element being rotationally biased relative to the plunger rod from the blocking position to the release position by a biasing member connecting the guide element to the syringe.

The method may comprise the biasing member being resiliently deformed as the plunger rod moves from the initial position to the intermediate position.

The method may comprise the biasing member being resiliently deformed when the plunger rod is in the initial position.

The method may comprise the guide element being free to move under the action of the biasing member to the release position when the follower enters the transition section of the guide track.

The method may comprise the guide element and the plunger rod being manually moveable between the blocking position and the release position.

The method may comprise substantially half of the medicament in the chamber being expelled as the plunger rod moves over the first range of motion, and substantially half of the medicament in the chamber being expelled as the plunger rod moves over the subsequent range of motion.

The method may comprise expelling/delivering more than two doses of medicament.

The method may comprise expelling/administering an RSV vaccine.

The delivery system may comprise an intranasal atomisation delivery device.

The RSV vaccine may be expelled/delivered intranasally with about ½ dose delivered to each nostril.

The intranasal dose of the RSV vaccine may be delivered in about 0.2 mL, wherein about 0.1 mL is delivered to each nostril.

The method may comprise intranasal atomization delivery of an average droplet size Dᵥ₅₀ of about 10-120 μm.

The method may comprise intranasal atomization delivery of an average droplet size Dᵥ₅₀ delivered to each nostril may be about 10-120 μm, about 30-110 μm, about 50-110 μm, about 70-110 μm, or about 80-110 μm.

The method may comprise intranasal atomization delivery so that an average shot volume to each nostril is between about 85 μL to about 120 μL, about 90 μL to about 115 μL, or about 95 μL to about 115 μL.

These and other aspects of the disclosure will be apparent from and elucidated with reference to the embodiments described hereinafter.

### BRIEF DESCRIPTION OF THE FIGURES

Embodiments will now be described, by way of example only, with reference to the accompanying drawings, in which:

FIG. 1 is a perspective view of a medicament delivery device of a first embodiment, shown in an initial position;

FIG. 2 is a side view of the device of FIG. 1;

FIG. 3 is a side view of the device of FIG. 1 rotated about 90 degrees from the view of FIG. 2

FIG. 4 is a side view of the device of FIG. 1 as shown in FIG. 3 but with the plunger rod removed;

FIG. 5 is a cross-sectional view along the line A-A of FIG. 3

FIG. 6 is a cross-sectional view along the line B-B of FIG. 4

FIG. 7 is a side view similar to FIG. 2 showing the device in a starting condition before use with the plunger rod in an initial position;

FIG. 8 is a side view of the device as shown in FIG. 7 after the plunger rod has been moved over a first range of motion into an intermediate position and a guide element is in a blocking position;

FIG. 9 is a side view of the device of FIG. 8 when the guide element has been rotated into a release position;

FIG. 10 is a side view of the device of FIG. 9 after the plunger rod has been moved over a second range of motion into an end position;

FIG. 11 is a perspective view of the guide element of the device of FIGS. 1 to 10;

FIG. 12 is a perspective view of the biasing member of the device of FIGS. 1 to 10;

FIG. 13 is a perspective view of the plunger rod of the device of FIGS. 1 to 10;

FIG. 14 is a side view of a medicament delivery device of a second embodiment in a starting condition with the plunger rod in an initial position;

FIG. 15 is a side view of a medicament delivery device of a third embodiment in a starting condition with the plunger rod in an initial position;

FIG. 16 is a side view of a medicament delivery device of a fourth embodiment in a starting condition with the plunger rod in an initial position;

FIG. 17 is an exploded perspective view of an apparatus comprising a guide element, biasing member, plunger rod and finger flange attachment of an embodiment, together with a portion of a syringe to which they connect;

FIG. 18 is a cross-sectional view of the apparatus of FIG. 17; and

FIG. 19 is a perspective view of a guide element of another embodiment.

### DETAILED DESCRIPTION

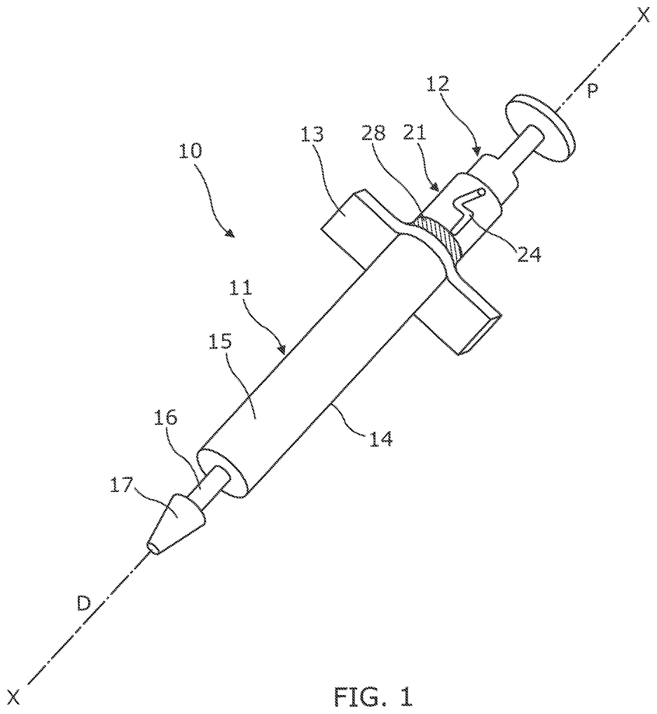

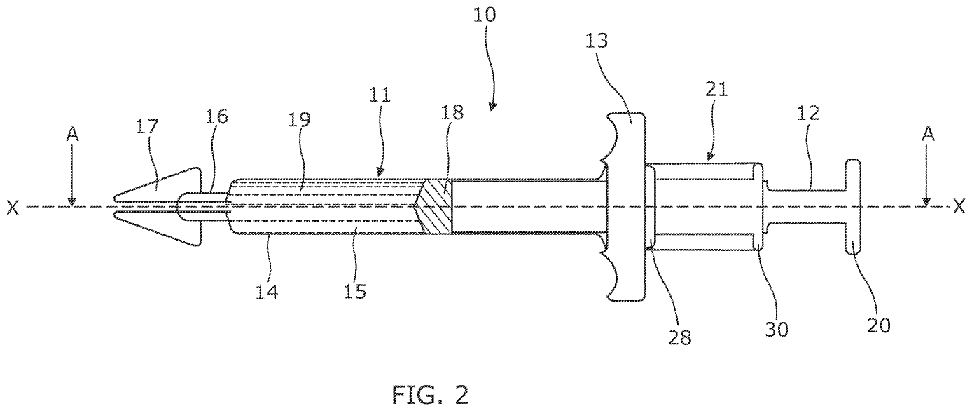

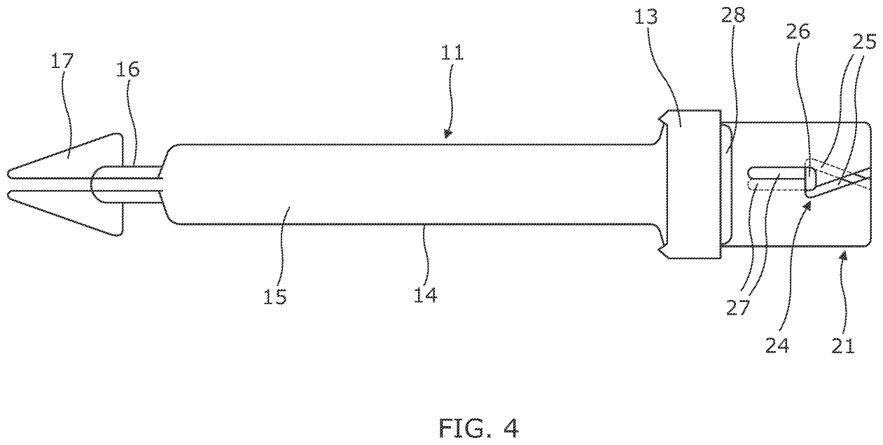

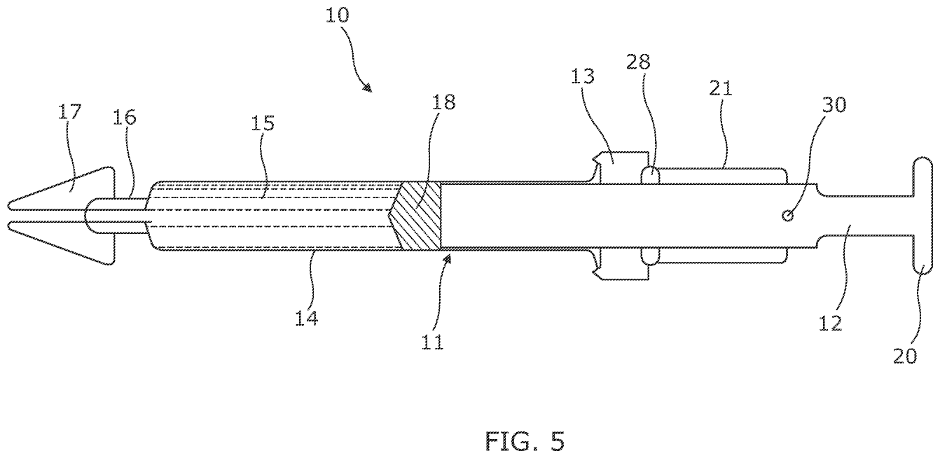

FIGS. 1 to 10 show a medicament delivery system or device 10 (hereafter “device 10”) according to a first embodiment. The device 10 has a proximal end P and a distal end D, and is generally elongate with a central axis X-X. The device 10 comprises a medicament container 11 in the form of a syringe (hereafter “container 11”), an actuator 12 in the form of a plunger rod 12, and finger flange 13 secured to a proximal end of the container 11. The finger flange 13 may be a separate component fixedly or detachably attached to the container 11, as described in more detail below. The container 11 is elongate and comprises an outer side wall 14 defining a medicament chamber 15. The container 11 is open at a proximal end P and includes an outlet 16 at the distal end D opposite to the proximal end of the container 11. The term “distal” refers to a location that is relatively closer to the outlet 16 and from which medicament is delivered in use, and the term “proximal” refers to a location that is relatively further away from the outlet 16. A distal region of the plunger rod 12, received within the container 11, is in sealing contact with the inside wall of the container 11. The proximal end of the plunger rod 12 includes an end surface 20 which, in use, a user would press a thumb against to dispense medicament (described in more detail below).

The outlet 16 comprises a narrowing of the container into a nozzle. An atomiser 17 is provided on the outlet 16. The atomiser 17 and/or outlet 16 may comprise a Luer lock to sealingly connect the atomiser 17 to the outlet 16. In some embodiments, the atomiser 17 may be formed to facilitate insertion of the outlet into an orifice into which medicament is to be delivered. Optionally, the atomiser 17 may be configured with a medicament passage configured to atomise a liquid medicament expelled through the outlet 16 and through the atomiser 17.

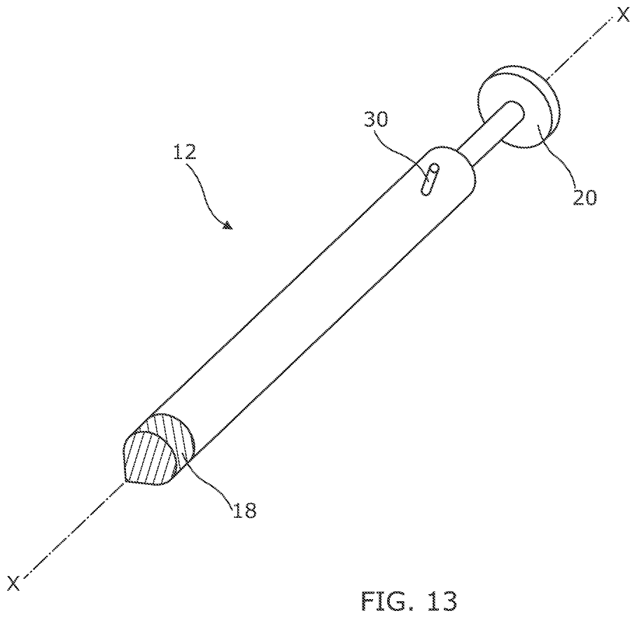

The container described herein may be any suitable shape or configuration, such as a hollow tube or other body. In the exemplary embodiment shown in FIGS. 1 to 10, the device 10 is generally in the form of a syringe where the container 11 comprises a syringe barrel and the plunger rod 12 is received within the syringe barrel. In some embodiments, a piston or plunger 18 may be provided within the medicament chamber 15 in sealing contact with the inside wall of the container 11. In such an arrangement, the piston 18 may be attached to the distal end of the plunger rod 12 (see FIG. 13), and the plunger rod 12 may therefore not need to make sealing contact with the inside wall of the container 11.

In use, medicament 19 would be provided within the medicament chamber 15, and would remain in the chamber 15 due to the seal of the plunger rod 12 and/or piston 18 at the proximal end of the chamber 15, and the narrow outlet 16 at the distal end of the chamber 15, which may be provided with a cap, valve, temporary rupturable seal, or other closure. Alternatively, the narrow outlet 16 and piston 18 seal alone may prevent medicament 19 escaping from the distal end of the chamber 15. The medicament 19 may be expelled in one or more doses from the chamber 15 by linearly moving the plunger rod 12 in a distal direction into the chamber 15 to reduce the volume of the chamber and force the medicament 19 out of the outlet 16.

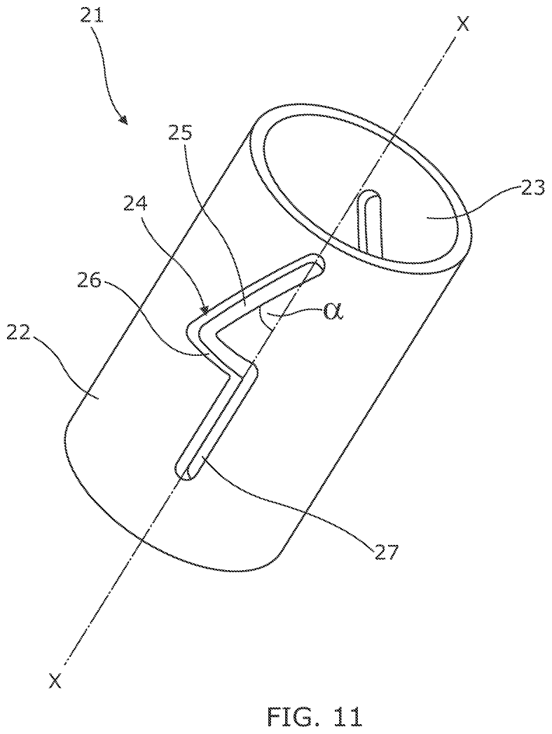

The device 10 includes a guide element 21 which functions as a “dose divider” in use of the device. The guide element 21 is in the form of a sleeve or collar, and may be a substantially cylindrical or tubular component. The guide element, sleeve, collar, or dose divider 21 (“guide element” hereafter) is shown in more detail in FIG. 11 and comprises a body 22 with an aperture 23 extending through the body 22. A guide track in the form of a slot 24 is formed in the side wall of the body 22. In the exemplary embodiment shown, two guide slots 24 are formed in the side wall of the guide element at diametrically opposite positions, although in other embodiments, one guide slot 24 or more than two guide slots 24 may be provided. The guide track may comprise a slot or groove extending partially through the thickness of the wall of the guide element, or may extend entirely through the thickness of the wall of the guide element.

The guide slot 24 comprises a first section 25, a second section 27, and a transition section 26 which connects the first and second sections 25, 27. The first section 25 is disposed at an acute angle α relative to the central longitudinal axis X-X of the device 10/guide element 21. The second section 27 is disposed substantially parallel to the central longitudinal axis X-X of the device 10/guide element 21. The transition section 26 connects the distal end of the first section 25 and the proximal end of the second section 27. The transition section 26 extends in a circumferential direction substantially perpendicular to the central longitudinal axis X-X of the device 10/guide element 21.

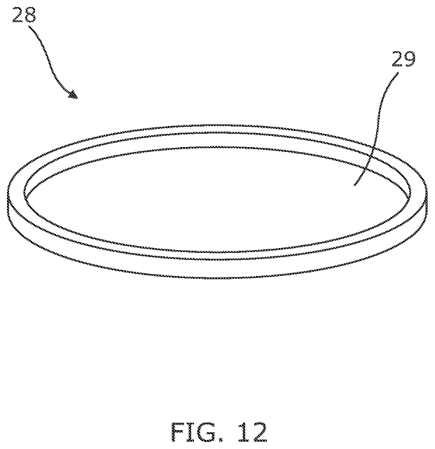

The guide element 21 is secured to the container 11 in the region of the finger flange 13 by means of a biasing member 28 (shown in detail in FIG. 12). In the exemplary embodiment shown, the biasing member 28 is a resiliently deformable component such as an elastomeric ring, with a central aperture 29. However, alternative configurations of biasing members are envisaged in alternative embodiments, such as a coil or torsion spring. The biasing member 28, guide element 21 and container 11/finger flange 13 are secured together by any appropriate means, such as a mechanical fastening or adhesive bonding. The resilience of the biasing member 28 is such that the guide element 21 can move rotationally to a limited degree relative to the container 11 about the longitudinal axis X-X of the device 10/guide element 21. The guide element 21 is rotationally moveable from a rest position in which the biasing member 28 is substantially undeflected or undeformed, to a deflected position in which the biasing member 28 has been deformed or deflected. In the deflected position, the resilience of the biasing member 28 rotationally urges the guide element 21 towards the rest position.

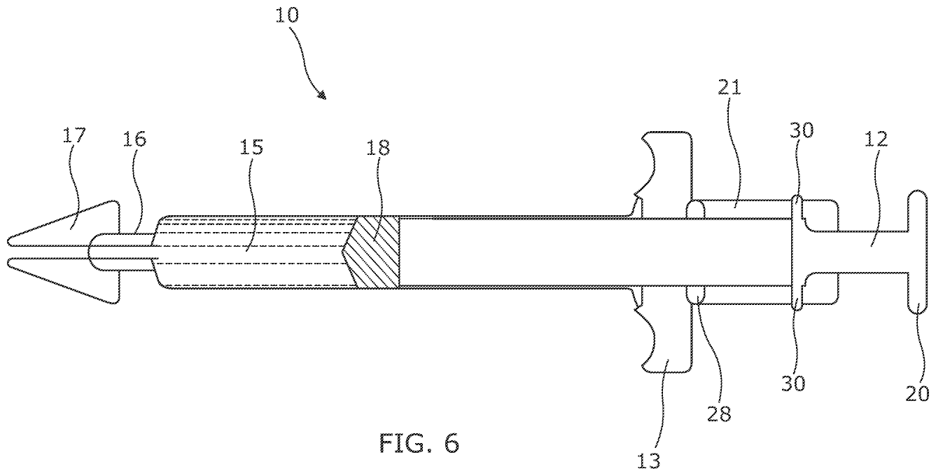

The plunger rod 12 is shown in more detail in FIG. 13 and comprises an elongate rod, with a piston or plunger 18 attached to the distal end so the plunger rod 12 serves as a piston rod or plunger rod for moving the piston 18 within the container 11. In various embodiments, the piston 18 may be formed integrally with the plunger rod 12, or may be mechanically or otherwise (such as by adhesive bonding) attached to the plunger rod 12, or the plunger rod 12 may abut against the piston 18. The plunger rod 12 comprises a follower in the form of a projection, such as a guide pin 30 projecting radially outwardly from the plunger rod 12 in a region near the proximal end P of the plunger rod 12. In the exemplary embodiment shown, the plunger rod 12 comprises two guide pins 30 disposed diametrically opposite to each other about the plunger rod 12, as can be seen in FIG. 6. However, in other embodiments, one guide pin 30 or more than two guide pins 30 may be provided. Each follower or guide pin 30 is received in a respective guide slot 24 in the guide element 21. The end surface 20 is disposed at the proximal end of the plunger rod 12, opposite to the piston 18.

In the exemplary embodiment shown in FIG. 13, the plunger rod 12 is substantially circular in cross-section. However, it is intended that other configurations of plunger rod may be provided and which may have different cross-sectional configurations, such as oval, square, triangular, hexagonal, or other polygonal shapes.

The plunger rod 12 is arranged to slide through the aperture 23 in the guide element 21 once the guide element 21 is secured to the container 11/finger flange 13 by means of the biasing member 28 as described above. When the guide element 21 is secured to the container 11/finger flange 13, the aperture 23 is aligned with the longitudinal axis X-X of the device 10 and of the bore of the medicament chamber 15, so the plunger rod 12 extends through the aperture 23 and into the container 11. The aperture 23 and plunger rod 12 are configured such that the plunger rod 12 can freely slide though the aperture 23 with the guide pins 30 in engagement with the guide slots 24.

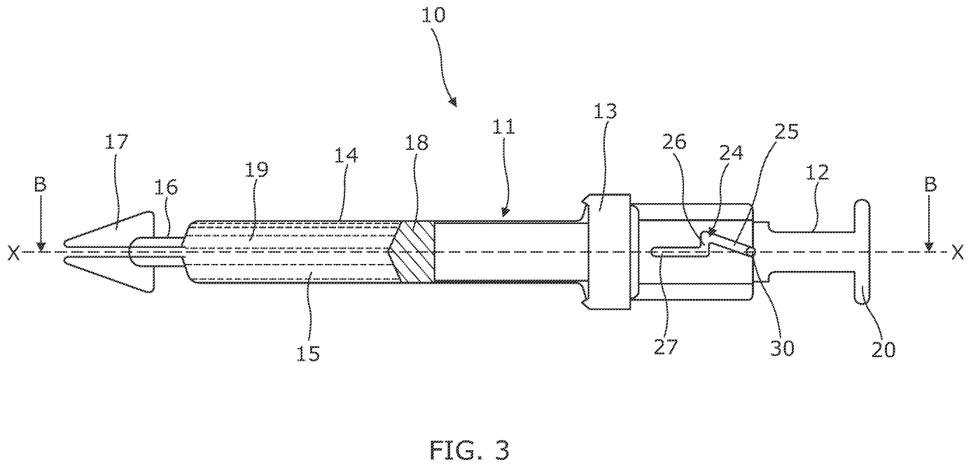

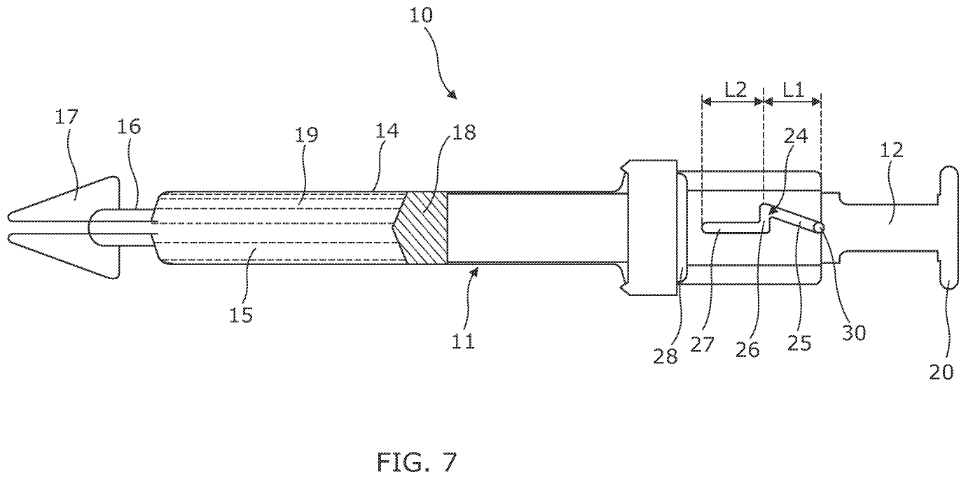

Operation of the device 10 of the above-described embodiment will now be described. The operation of the device 10 will be described in the context of use as an intranasal medicament delivery device 10, that is, a device for delivery of medicament into the nostril(s) of a patient. However, the concept is not intended to be limited to intranasal devices and may equally be applicable to various other forms of medicament delivery devices in which the medicament is intended to be delivered in two or more separate doses. In the context of an intranasal device, the volume of medicament in the device 10, for example a vaccine, is often required to be delivered into each nostril, often with the volume of medicament divided equally into a dose for each nostril. In use of such a device, the device 10 starts in a starting condition shown in FIGS. 3 and 7. Here, the plunger rod 12 is in an initial position and none of the medicament 19 has been expelled from within the chamber 15. Also in this initial position, the guide pins 30 are located at the proximal ends of the respective first section 25 of each guide slot 24, and the guide element 21 is in a rest position, being substantially unbiased by the biasing member 28.

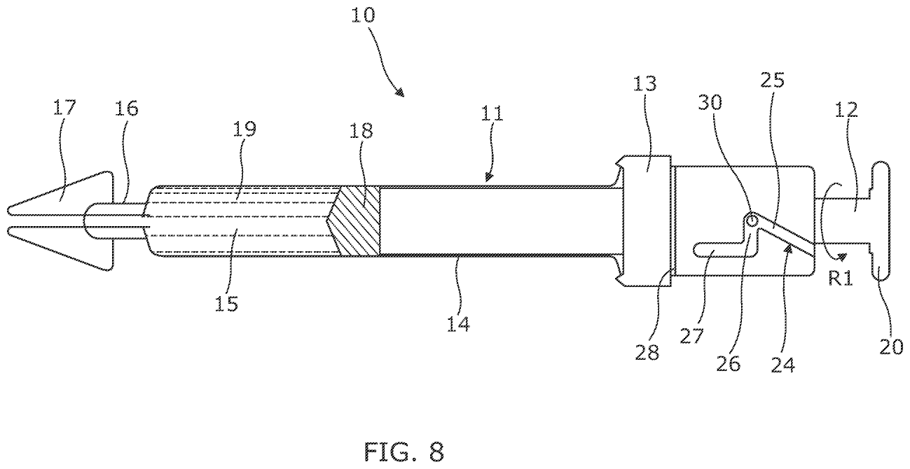

The user (which may be a patient them self if self-administered, or a healthcare professional or other user if the medicament is being administered to the patient by a third party) inserts the atomiser 17 into the first nostril. The user then presses the end surface 20 with their thumb, with their index and middle fingers on the finger flange 13, to move the plunger rod 12 axially in a distal direction D. This pushes the piston 18 distally within the chamber 15 and expels the medicament 19 out of the outlet 16 and through the medicament passage of the atomiser 17 to atomise the medicament for inhalation by the patient. As the plunger rod 12 is moved distally, the guide pins 30 travel distally along the respective first section 25 of the guide slots 24. The user's hold on the finger flange 13 and friction of their thumb on the end surface substantially prevents relative rotation between the plunger rod 12 and the container 11. Therefore, since the first section 25 of the guide slot 24 is oriented at an acute angle α relative to the longitudinal axis X-X of the device, the guide element 21 is caused to rotate about the longitudinal axis X-X, as shown by arrow R1 in FIG. 8. The user continues to move the plunger rod 12 distally over this first range of motion, rotating the guide element 21 as described above, until the guide pin 30 abuts a proximal wall of the transition section 26 of the guide slot 24 and the plunger rod 12 therefore cannot move any further in the distal direction. At this point, the plunger rod 12 is in an intermediate position, the guide element 21 is in a rotationally deflected position in which it is in a blocking position and a first dose of the medicament 19 has been delivered to the patient. This is shown in FIG. 8. Also at this point, in some embodiments, it may be that pressure or friction of the guide pins 30 against the respective wall of the transition section 26 of the guide slot 24 is such that the guide element 21 is prevented from rotating back to the rest position under the force of the biasing member 28. In other embodiments, it may be that the movement of the plunger rod 12 over the first range of motion is such that the guide pins 30 abut the proximally-facing wall of the transition section 26 of the guide slot 24 and thereby indicates to a user that the first dose of medicament has been delivered, before the guide element 21 is able to rotate back to the rest position under the force of the biasing member 28., This may be by selection of an appropriate material and dimension of the biasing member 28 to produce an appropriate resistance to deformation and thereby rotation of the guide element 21, and when in the deflected position, a predetermined rotational force is exerted on the guide element 21 by the biasing member 28.

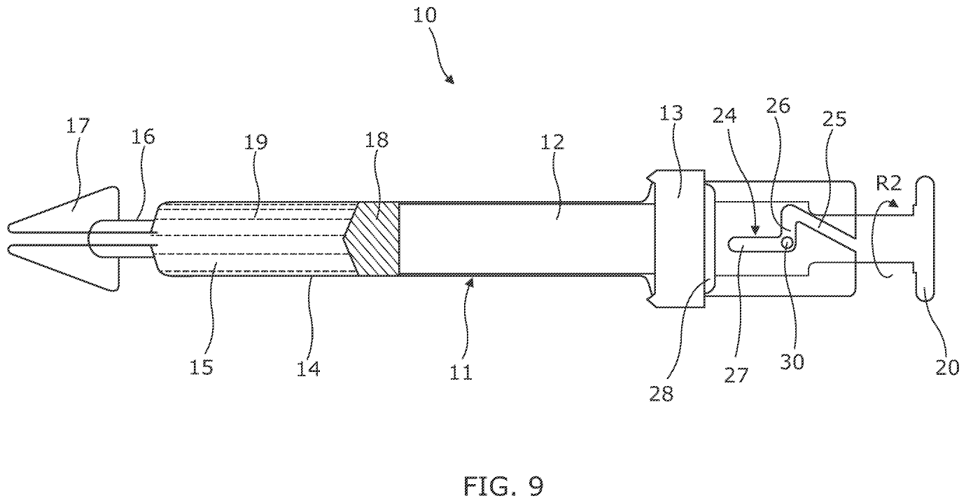

After the user has delivered the first dose of medicament, they stop exerting force on the end surface 20 of the plunger rod 12, and guide element 21 is able to rotate back to the start position under the biasing force of the deflected biasing member 28, as shown by arrow R2 in FIG. 9. As the guide element 21 rotates relative to the container 11 under the force of the biasing member 28, the guide pins 30 travel along the respective transition section 26 from the distal end of the first section 25 until they reach the end of the transition section 26 at the proximal end of the second section 27. At this point, the guide element 21 is in a release position shown in FIG. 9 and further axial movement of the plunger rod 12 is no longer prevented by the guide pins 30 abutting the proximal wall of the respective transition section 26 of the guide slot 24.

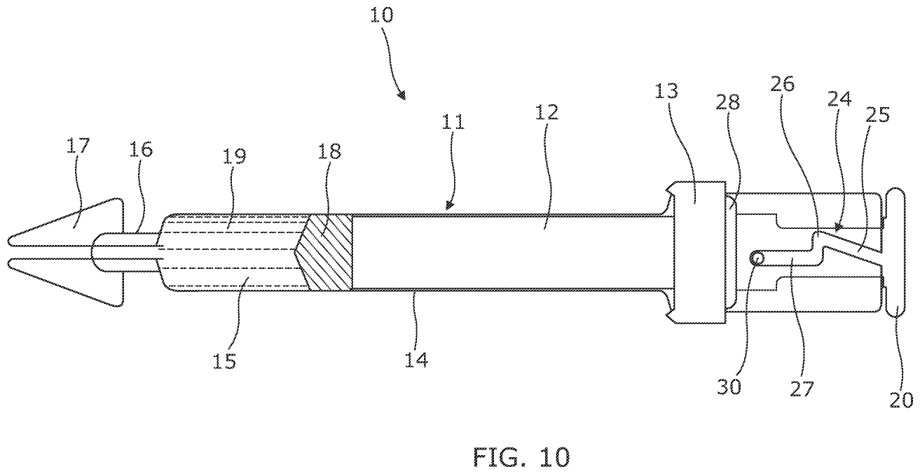

The user then removes the device 10 from the first nostril and then inserts the atomiser 17 into the other nostril. Since the guide element 21 is in the release position, the plunger rod 12 is no longer prevented from moving further axially in the distal direction. The user then presses the end surface 20 again to move the plunger rod 12 axially in a distal direction D which pushes the piston 18 distally within the chamber 15 and expels a further dose of medicament 19 out of the outlet 16 and through the medicament passage of the atomiser 17 to atomise the medicament for inhalation by the patient. The user continues to move the plunger rod 12 distally over this second range of motion until either the distal face of the end surface 20 abuts the proximal end of the guide element 21 or the guide pins 30 abut the distal end of the respective second section 27 of the guide slot 24. This may coincide with the piston 18 abutting the distal end of the chamber 15. At this point, the plunger rod 12 is in an end position as shown in FIG. 10 and the second dose of medicament 19 has been expelled and delivered to the patient.

The device 10 described above provides a simple and effective way to ensure a user is able to divide the medicament delivery process into two separate doses, and the guide element 21 provides an effective way to ensure the medicament 19 is divided as intended and the user cannot accidentally deliver too much/all of the medicament in the first dose. Furthermore, since the guide element 21 is connected to the container 11 by a biasing member 28 and is resiliently rotationally deflected, the device 10 of the first embodiment provides an automatic transition between a blocking position after the first dose has been delivered, to a release position ready for the second dose to be delivered. This further helps towards ensuring the medicament is delivered in the intended dose volumes in each dose and provides enhanced usability of the device 10.

As can be seen from FIG. 7, the distance between the proximal end and the distal end of the first section 25 of the guide slot 24 has a length dimension in the axial direction of the device 10 of L1. The distance between the proximal end and the distal end of the second section 27 of the guide slot 24 has a length dimension in the axial direction of the device 10 of L2. It will be appreciated that the volume of medicament 19 delivered in the first dose is determined by the distance the plunger rod 12 is able to move from the initial position (shown in FIG. 7) to the intermediate position (shown in FIGS. 8 and 9). This is limited by when the guide pins 30 abut the respective proximal wall of the transition section 26 at the distal end of the first section 25 of the guide slot 24. That is, this volume of medicament of the first dose is determined by the distance L1 over which the plunger rod 12 moves in the first range of motion. It will also be appreciated that the volume of medicament 19 delivered in the second dose is determined by the remaining distance the plunger rod 12 is able to move from the intermediate position (shown in FIGS. 8 and 9) to the end position (shown in FIG. 10). This is limited by when the guide pins 30 abut the respective distal end of the second section 27 of the guide slot 24. This may also coincide with when the distal face of the end surface 20 abuts the proximal end of the guide element 21, and/or when the piston 18 abuts the distal end of the chamber 15. That is, this volume of medicament of the second dose is determined by the distance L2 over which the plunger rod 12 moves in the second range of motion.

In view of the above, the ratio of volumes of first and second doses of medicament to be delivered can be dictated by controlling the first and second ranges of motion of the plunger rod 12, which may be controlled by selecting dimensions L1 and L2 of the device 10 as necessary by appropriate dimensioning of the first and second sections 25, 27 of the guide slot 24. In embodiments where the first and second doses are to be equal, it will be appreciated that dimensions L1 and L2 can be made the same.

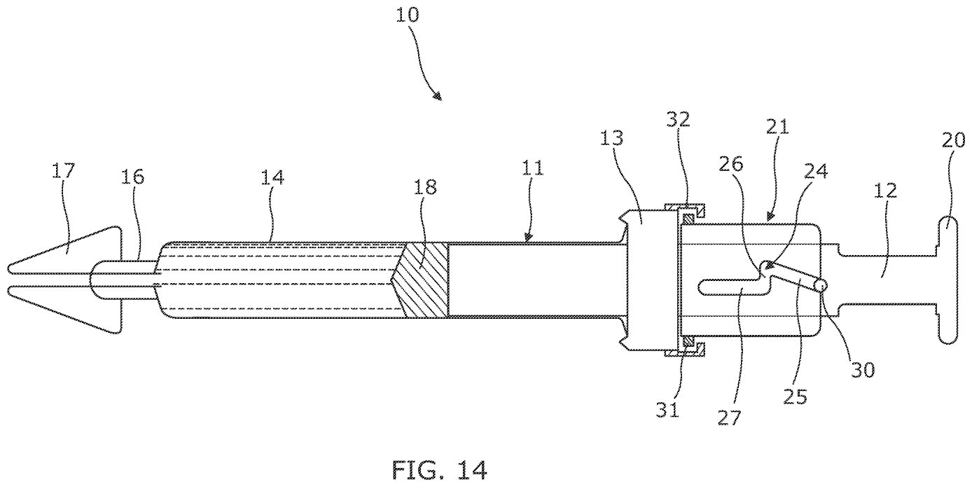

FIG. 14 is a side view a medicament delivery device 10 according to a second embodiment. The device 10 of the second embodiment has a number of features in common with the device 10 of the first embodiment. These like features retain the same reference numerals and will not be described in detail again.

A difference with the device 10 of the second embodiment is that there is no biasing member 28. Since the guide element 21 is not secured to the container 11 by such a biasing member 28, the guide element 21 is not rotationally biased relative to the container 11. In one arrangement, the guide element 21 is rotatable around the plunger rod 12 and retained in place by virtue of the plunger rod 12 extending through the aperture 23 in the guide element 21 and the guide pins 30 received in the guide slots 24. In an alternative arrangement, the guide element 21 may be coupled to the container 11 by a retaining mechanism configured to allow rotational movement of the guide element 21 relative to the container 11, and constrain movement of the guide element 21 in a axial direction X-X relative to the container 11. Such an arrangement is shown in FIG. 14 in which the retaining mechanism comprises one or more projections 31 extending radially from an outer surface of the guide element 21. The container 11 or finger flange 13 comprises one or more retaining elements 32 which extend around and hook over the projections 31. The projection(s) 31 may comprise a single annular flange around part or all of the perimeter of the guide element 21, or a plurality of projections around the perimeter of the guide element 21. The retaining element(s) 32 may comprise a single annular lip or hook around the perimeter of the container 11/finger flange 13, or a plurality of lips or hooks around the perimeter of the container 11/finger flange 13. The retaining element(s) 32 extend over the projection(s) 31 so as to have a portion that is disposed proximally in an axial direction X-X of the projection(s) 31 to constrain axial movement of the guide element 21 relative to the container 11.

In use of the device 10 of the second embodiment, the operation would be similar to that described above with reference to the first embodiment. However, in the absence of the biasing member 28, the guide element 21 is not biased back towards a start position as the guide pins 30 travel along the first, inclined section 25 of the guide slot 24 and the guide element 21 is rotated to the blocking position. Therefore, when the plunger rod 12 is in the intermediate position, the guide element 21 is not urged back towards the release position. Accordingly, once the first dose of medicament has been delivered and the plunger rod 12 is in the intermediate position and is prevented from further axial movement by the guide element 21 being in the blocking position, the user is required to manually rotate the guide element 21 into the release position in order to permit the second dose of medicament to be delivered. Thereafter, the function of the device 10 of the second embodiment is the same as described above with respect to the first embodiment. However, the device 10 of the second embodiment is still configured to provide the dose-dividing benefits of the first embodiment described above and is simple to use. The absence of biasing member 28 may also make construction of the device simpler using fewer parts.

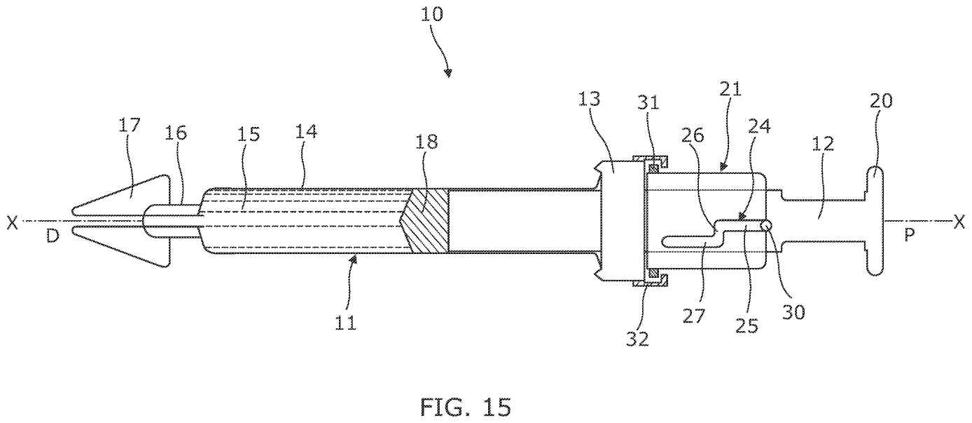

FIG. 15 is a side view a medicament delivery device 10 according to a third embodiment. The device 10 of the third embodiment has a number of features in common with the device 10 of the second embodiment, which retain the same reference numerals and will not be described in detail again.

As with the device 10 of the second embodiment, the device 10 of the third embodiment also omits the biasing member 28. A difference with the device 10 of the third embodiment over that of the second embodiment (and first embodiment) is the configuration of the guide slot 24. In the device 10 of the first and second embodiments, first section 25 is disposed at an acute angle α relative to the central longitudinal axis X-X of the device 10/guide element 21. The transition section 26 and second section 27 are configured in the same manner as those features of the first and second embodiments. However, the first section 25 of the guide slot 24 is arranged substantially parallel to the central longitudinal axis X-X of the device 10/guide element 21 and parallel to the second section 27 of the guide slot 24. Accordingly, as the plunger rod 12 is moved over the first range of motion from the initial position to the intermediate position, the guide element 21 is not caused to rotate about the longitudinal axis X-X

As with the device 10 of the second embodiment, the guide element 21 is not rotationally biased relative to the container 11 and may be retained in place by virtue of the plunger rod 12 extending through the aperture 23 in the guide element 21 and the guide pins 30 received in the guide slots 24, or may comprise a retaining mechanism in any of the configurations described above.

In use of the device 10 of the third embodiment, the operation would be similar to that described above with reference to the first embodiment. However, since the first section 25 of the guide slot 24 extends substantially parallel to the longitudinal axis X-X of the device 10, the guide element 21 is not rotated as the guide pins 30 travel along the first section 25 of the guide slot 24. However, when the guide pins 30 reach the distal end of the respective first section 25 and abut the proximal wall of the transition section 26, the guide element 21 is still in a blocking position and further axial movement of the plunger rod 12 is prevented. The guide element 21 is not urged back towards the release position as there is no biasing member 28 in the third embodiment. Accordingly, once the first dose of medicament has been delivered and the plunger rod 12 is in the intermediate position, the user is required to manually rotate the guide element 21 into the release position in order to permit the second dose of medicament to be delivered. Thereafter, the function of the device 10 of the third embodiment is the same as described above with respect to the first and second embodiments. The device 10 of the third embodiment is still configured to provide the dose-dividing benefits of the first and second embodiments described above and is simple to use.

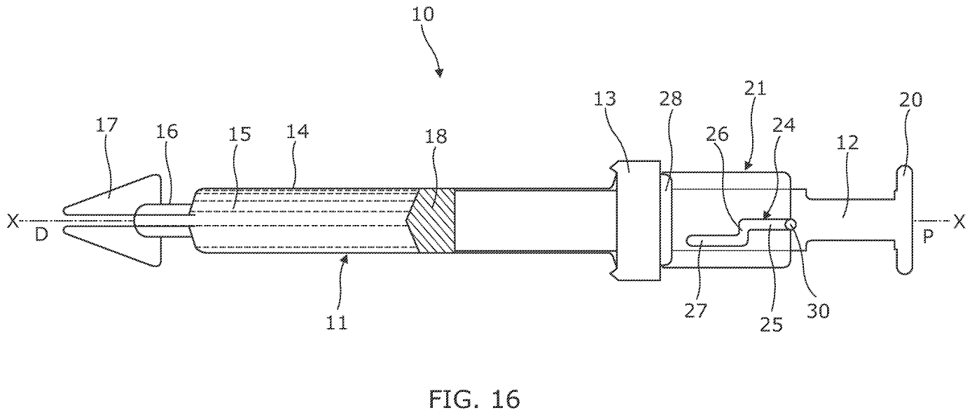

FIG. 16 is a side view a medicament delivery device 10 according to a fourth embodiment. The device 10 of the fourth embodiment has a number of features in common with the devices 10 of the first and third embodiments, which retain the same reference numerals and will not be described in detail again.

As with the device 10 of the third embodiment, the first section 25 of the guide slot 24 is disposed substantially parallel to the longitudinal axis X-X of the device 10/guide element 21. The transition section 26 and second section 27 are configured in the same manner as those features of the first to third embodiments. Accordingly, as the plunger rod 12 is moved over the first range of motion from the initial position to the intermediate position, the guide element 21 is not caused to rotate about the longitudinal axis X-X. However, unlike the third embodiment, the fourth embodiment includes a biasing member 28 which may be of any arrangement described above with respect to the device 10 of the first embodiment.

In use of the device 10 of the fourth embodiment, the operation would be similar to that described above with reference to the first embodiment. However, the guide element 21 may be rotationally biased relative to the container 11/plunger rod 12/finger flange 13 when the plunger rod 12 is in the initial position and the device is in the starting condition. That is, the biasing member 28 may be in a deflected or stressed/deformed state when the device 10 is in the starting condition. In order that the force of the deflected biasing member 28 does not rotate the plunger rod 12 within the container 11 before use of the device 10 and move into a relaxed state, there may be anti-rotation features between the plunger rod 12 and container 11. For example, one or more elongate ribs may be formed on the plunger rod 12 which are received in corresponding groove(s) formed on the container 11 or finger flange 13, or vice versa. Alternatively, the plunger rod 12 and container 11 may be non-circular in cross-section, for example oval, square, rectangular, etc. Thereby, relative rotation between the plunger rod 12 and container 11 would be prevented.

The plunger rod 12 is moved over the first range of motion to dispense the first dose of medicament. The guide pins 30 travel along the first section 25 of the guide slot 24 although the guide element 21 is not rotated during this movement of the plunger rod 12. When the guide pins 30 reach the distal end of the respective first section 25 and abut the proximal wall of the transition section 26, the guide element 21 is in a blocking position and further axial movement of the plunger rod 12 is prevented. However, since the guide element 21 is initially rotationally biased relative to the container 11/plunger rod 12 by the biasing member 28, once the first dose of medicament has been delivered and the plunger rod 12 is in the intermediate position, the user is not required to manually rotate the guide element 21 into the release position. Instead, the guide element 21 automatically rotates under the action of the deflected biasing member 28 into the release position. The guide pins 30 travel along the respective transition section 26 until located at the proximal end of the respective second section 27. Thereafter, the function of the device 10 of the fourth embodiment is the same as described above with respect to the first to third embodiments. The device 10 of the fourth embodiment is still configured to provide the dose-dividing benefits of the first to third embodiments described above and is simple to use, and also avoids the requirement for the user to manually move the guide element 21 into the release position once the first dose of medicament has been delivered. In a variant of the fourth embodiment, the biasing member 28 may not be initially rotationally biased and may be in a relaxed state in the start position. In such an embodiment, a user would then rotate the guide element 21 into the release position against the biasing force of the biasing member 28 to be able to dispense the second dose of medicament.

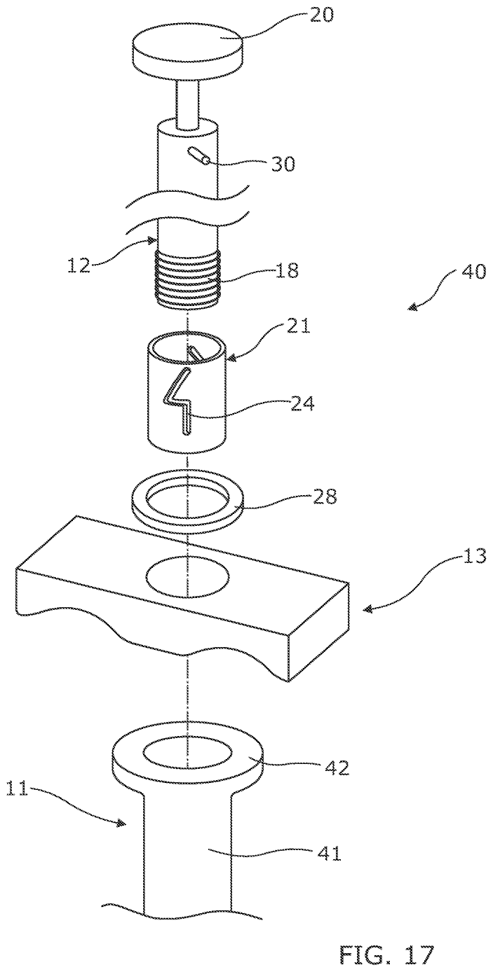

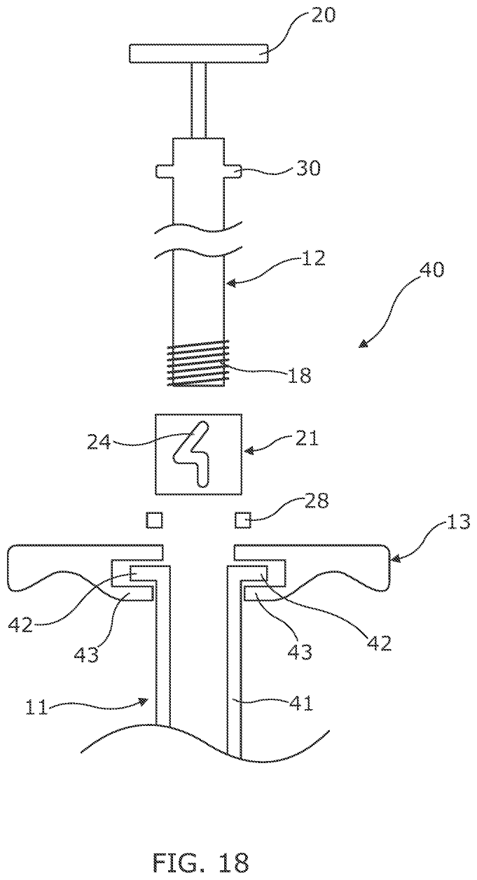

FIGS. 17 and 18 show an apparatus 40 of an embodiment, configured for attachment to a medicament container 11 such as a syringe. Like features from the previously-described embodiments retain the same reference numerals. The apparatus 40 comprises a finger flange 13, a guide element 21, a biasing member 28 and a plunger rod 12, all as previously described with respect to the first embodiment. It is intended that the apparatus 40 can be attached to a standard syringe having a barrel 41 and a lip 42 projecting outwardly around the edge of the barrel 41 at the proximal end thereof. Thereby, the apparatus 40 can be supplied as a separate kit to be fitted to conventional syringes, or retro-fitted to such syringes, before or after assembly and/or filling with a medicament. The components are shown in exploded view in FIG. 17 for illustration, although may be supplied connected, such as with the finger flange 13, biasing member 28 and guide element 21 attached, such as adhesively bonded. The plunger rod 12 may be supplied fitted through the aperture 23 of the guide element 21 with the pins 30 located in a respective guide slot 24.

The finger flange 13 includes projections 43 (see FIG. 18) which can locate distally of the lip 42 to secure the finger flange 13 to the medicament container/syringe 11. Thereafter, once secured to a medicament container/syringe 11, operation of the medicament delivery device 10 is as previously described, so will not be repeated. It will be appreciated that the apparatus 40 for fitting to a container such as a syringe, may be configured as described above with reference to any of the first to fourth embodiments, such as omitting the biasing member 28, including the projections 31 on the guide element 21 and retaining elements 32 on the finger flange 13, and/or with different shaped guide slots 24 as described herein.

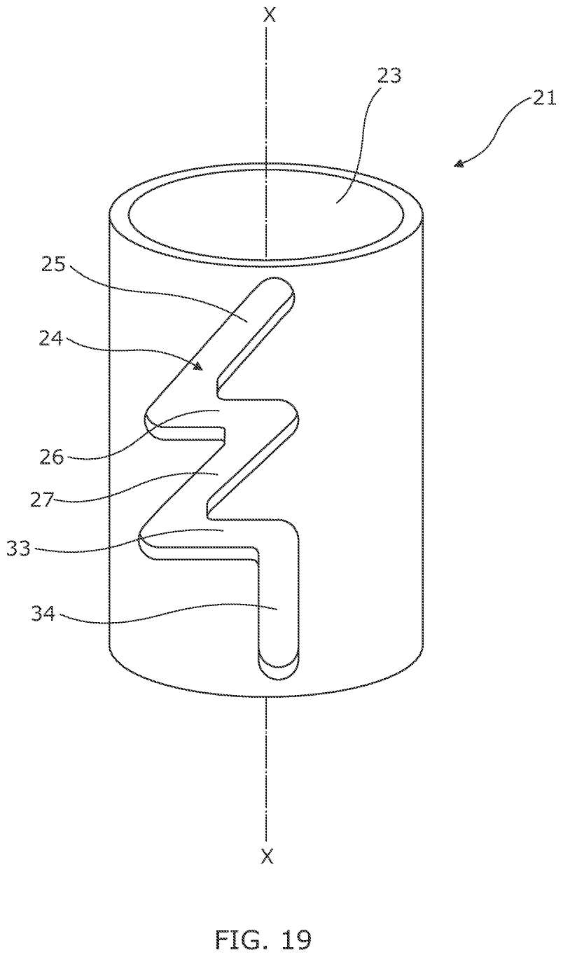

FIG. 19 shows a guide element 21 of another embodiment. As mentioned above, medicament delivery devices/apparatuses of the present disclosure can enable a medicament dose to be divided into two or more doses. The embodiments described above allow a medicament to be divided into two doses. However, the present disclosure is not intended to be limited to a medicament being divided into two doses, and may include a medicament being divided into more than two doses. A guide element 21 useable in place of the guide element 21 of the first embodiment is shown in FIG. 19. Like features retain the same reference numerals. A difference of the guide element of FIG. 19 is that the guide slot 24 includes a first section 25, a second section 27 connected by the transition section 26, but also includes a third section 34 which is connected to the second section 27 by a second transition section 33. Furthermore, the second section 27 is inclined relative to the axial direction, as per the first section 25, and the third section 34 extends in a direction substantially parallel to the axial direction.

It will be appreciated that when the guide element 21 of FIG. 19 is used in the medicament delivery device 10 of FIG. 1, the medicament would be divided into three doses, respectively deliverable as the plunger rod 12 is moved axially and the guide pins 30 move along the respective first, second, and third sections (25, 27, 34) of the guide slot 24. As the second section 27 is inclined, the biasing member 28 is also rotationally biased as the second dose of medicament is delivered as the guide pins 30 move along the second section 27 of the guide slot 24. Furthermore, the guide element 21 is not rotationally biased as the third dose of medicament is delivered since the third section 34 of the guide slot 24 extends substantially axially.

It will be appreciated that more than two or three doses of medicament may be delivered by appropriately forming the guide slot 24 to include an appropriate number of sections extending at least partially in an axial direction (i.e. at an inclined angle or parallel to the axial direction), connected by appropriate transition sections. For example, a medicament may be delivered in four or more doses within the scope of the present disclosure.

The proximal end of the first section 25 of the guide slots 24 may be closed, or in alternative embodiments, may be open. In embodiments where the proximal end of the first sections 25 are closed, this may help prevent accidental removal of the plunger rod 12 from the container 11 in the proximal direction before use which could render the device 10 unusable. In embodiments where the proximal end of the first sections 25 are open, this may aid ease of manufacture for the guide pins 30 of the plunger rod to be inserted into the guide slots 24 during assembly of the device 10.

In the embodiments described above, the follower(s) or guide pin(s) 30 are provided on the plunger rod 12 and the guide track(s) or guide slot(s) are provided on the guide element 21.

However, in an alternative arrangement, the follower(s)/guide pin(s) 30 may be provided on the guide element 21 and the guide track(s)/slot(s) may be provided on the plunger rod 12.

Described herein are systems, devices, components, and methods that may be associated with delivering a respiratory syncytial virus (RSV) vaccine using an intranasal atomization delivery device.

As used herein, “RSV ΔNS2/Δ1313/I1314L” refers to an RSV ΔNS2/Δ1313/I1314L (NIH) or an RSV ΔNS2/Δ1313/I1314L (Sanofi). Each of the ΔNS2/Δ1313/I1314L (NIH) and the RSV ΔNS2/Δ1313/I1314L (Sanofi) comprise a live-attenuated RSV with (i) a 523 nucleotide (nt) deletion of the NS2 gene (ΔNS2), (ii) an amino acid deletion in the L protein, and (iii) a genetically stabilizing mutation in the L gene. The live-attenuated RSV of the RSV ΔNS2/Δ1313/I1314L (Sanofi) also includes a nucleotide modification at position 14456 that represents a change from a thymine (T) to an adenine (A) in a non-coding region.

As used herein, “RSV ΔNS2/Δ1313/I1314L vaccine” refers to an “RSV ΔNS2/Δ1313/I1314L (NIH) vaccine” or an “RSV ΔNS2/Δ1313/I1314L (Sanofi) vaccine.” An RSV ΔNS2/Δ1313/I1314L (NIH) vaccine comprises an effective amount of RSV ΔNS2/Δ1313/I1314L (NIH). An RSV ΔNS2/Δ1313/I1314L (Sanofi) vaccine comprises an effective amount of RSV ΔNS2/Δ1313/I1314L (Sanofi).

### Exemplary Methods and Uses

In some embodiments, provided are methods of delivering a dose of an RSV vaccine using an intranasal atomization delivery device.

In some embodiments, the methods of administering a dose of an RSV vaccine that comprises a live attenuated RSV use an intranasal atomization delivery device. In some embodiments, provided are methods of administering a dose of an RSV vaccine using an intranasal atomization delivery device, the RSV vaccine comprising an effective amount of a live-attenuated RSV with (i) a 523 nucleotide (nt) deletion of the NS 2 gene (ΔNS2), (ii) an amino acid deletion in the L protein, and (iii) a genetically stabilizing mutation in the L gene (RSV ΔNS2/Δ1313/I1314L (NIH) vaccine) or RSV ΔNS2/Δ1313/I1314L (Sanofi) vaccine that further comprises a nucleotide modification in a non-coding region that represents a change from a thymine (T) to an adenine (A). In some embodiments, a codon that encodes a serine at position 1313 of the L protein is deleted resulting in the deletion of the amino acid in the L protein (Δ1313). In some embodiments, an amino acid residue substitution of leucine for isoleucine at position 1314 results in a genetically stabilizing mutation in the L gene (I1314L).

In some embodiments, provided are methods of administering a dose of an RSV vaccine using an intranasal atomization delivery device. In some embodiments, the paediatric subject may be 6 to 18 months of age.

In some embodiments, the RSV vaccine is delivered intranasally using the intranasal atomization delivery device to deliver about ½ dose to each nostril. In some embodiments, the RSV vaccine is delivered intranasally so that the whole dose is delivered to one nostril. In some embodiments, the RSV vaccine is delivered intranasally with ½ dose delivered to one nostril and the other ½ dose is delivered to the same nostril after the first ½ dose is absorbed. In some embodiments, the RSV vaccine is delivered intranasally using the intranasal atomization delivery device to deliver a dose in unequal amounts to one or both nostrils.

In some embodiments, the RSV vaccine is delivered intranasally using the intranasal atomization delivery device in a liquid formulation. In some embodiments, the RSV vaccine dose is delivered intranasally using the intranasal atomization delivery device in about 0.2 mL. In some embodiments, the 0.2 mL dose is delivered intranasally wherein about 0.1 mL is delivered to each nostril. In some embodiments, the RSV vaccine dose is delivered intranasally using the intranasal atomization delivery device in about 0.01 mL, 0.02 mL, 0.05 mL, 0.1 mL, 0.2 mL, 0.3 mL, 0.4 mL, 0.5 mL, 0.6 mL, 0.7 mL, 0.8 mL, 0.9 mL, 1.0 mL, 1.1 mL, 1.2 mL, 1.3 mL, 1.4 mL or 1.5 mL. As described above, the dose may be divided evenly between two nostrils, divided unevenly between two nostrils, or delivered all to one nostril in one or more deliveries.

In some embodiments, provided are methods of administering a first dose of an RSV vaccine using an intranasal atomization delivery device and administering a second dose of the RSV vaccine using an intranasal atomization delivery device.

In some embodiments, provided are methods of administering a first dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, provided are methods of administering a dose of an RSV vaccine using an intranasal atomization delivery device wherein the intranasal atomization delivery device comprises a spray nozzle to atomize the RSV vaccine and direct a spray plume toward a top of a nasal passageway into a nasal cavity. In some embodiments, the intranasal atomization delivery device comprises a barrel operably connected to the spray nozzle and a plunger movable within the barrel to advance the RSV vaccine through the spray nozzle. In some embodiments, the intranasal atomization delivery device further includes a dose divider for splitting the dose of the RSV vaccine into two or more deliveries. In some embodiments, the dose divider may divide the dose of the RSV vaccine in about half of a volume to be delivered to a subject. In some embodiments the dose divider is used to deliver ½ dose to each nostril of the subject.

In some embodiments, the intranasal atomization delivery device delivers an average droplet size Dᵥ₅₀ of 10-120 μm. In some embodiments, the intranasal atomization delivery device delivers an average droplet size Dᵥ₅₀ of 10-120 μm, about 30-110 μm, about 50-110 μm, about 70-110 μm, or about 80-110 μm. In some embodiments, the intranasal atomization delivery device delivers an average shot weight between about 95 mg to about 135 mg, between about 100 mg to about 130 mg, or between about 100 mg to about 130 mg or between about 105 mg to about 130. In some embodiments, the intranasal atomization delivery device delivers an average shot volume of about 85 μL to about 120 μL, about 90 μL to about 115 μL, or about 95 μL to about 115 μL.

In some embodiments, use of an intranasal atomization delivery device described herein for administering a dose of an RSV vaccine to a subject is provided wherein the RSV vaccine comprises an effective amount of a live-attenuated RSV. In some embodiments, the use includes a live-attenuated RSV with (i) a 523 nucleotide (nt) deletion of the NS2 gene (ΔNS2), (ii) an amino acid deletion in the L protein, and (iii) a genetically stabilizing mutation in the L gene (RSV ΔNS2/Δ1313/I1314L) (NIH) vaccine or RSV ΔNS2/Δ1313/I1314L (Sanofi) vaccine that further comprises a nucleotide modification in a non-coding region that represents a change from a thymine (T) to an adenine (A).

### Immunogenicity

Approximately seventy percent (70%) of vaccine recipients attained a 4-fold response in neutralizing antibody titer following the second vaccine dose in both low and high dose groups, compared to 61 and 47% following one vaccine dose in low and high dose RSV naïve participants. This increase in the percentage of participants attaining a 4-fold response following a second administration supports the use of a second vaccine administration in this population. The 70% attaining this fold rise post second vaccine administration aligns with the expected clinical efficacy goal of 70% for the candidate. After one or two vaccinations, 75% attained a 4-fold rise in serum neutralizing antibody titers. The percentage of RSV experienced participants (36% and 22% in the low and high dose groups respectively) also attaining a 4-fold response in neutralizing antibody titers post vaccination 1 suggests potential benefit for this sub-group as well. It must be noted that this comes from a modest sample of participants (n=20 of 97 vaccine recipients) at this point of interim analysis.

Taken together, these data strongly support the use of RSV ΔNS2/Δ1313/I1314L (Sanofi) vaccine at an operative range study for the candidate including doses at 5.6 and 6.2 log PFU/dose.

### Vaccine Virus Shedding and Infectivity

Following each vaccine administration, the shedding of vaccine virus was considered in addition to the fold rise in neutralizing antibody titers or serum IgG as vaccine infectivity. The high level of vaccine infectivity (over 80% and 70% in RSV naïve following the first and second vaccine administration respectively) is supportive of a promising vaccine candidate associated with good vaccine take. When infectivity was considered after either vaccine administration, over 90% of participants had evidence of infection. In the small cohort of RSV experienced participants, relatively high infectivity (80% and 33.3% in low and high dose recipients following one vaccine administration and 60 and 50% following a second vaccine administration) was found, implicating promise for this group. In addition, the marked drop in the percentage of vaccine virus shedders in RSV naïve participants after the second vaccine administration (roughly 20%) compared to the first vaccine administration (over 70%) is as previously documented with other efficacious live-attenuated mucosal viral vaccines where subsequent ‘challenge’ in the form of a second vaccine dose is characterized by a marked reduction in vaccine virus shedding. Of note, the shedding data available for this cohort was from data at a single timepoint following each vaccination (seven days post vaccination). While this coincides with the point of peak viral shedding documented in other RSV live-attenuated vaccine (LAV) trials, it is likely that some shedders may have been missed. This limitation in the available shedding data makes the results obtained particularly encouraging.

Interim analysis results showed a promising safety, immunogenicity and infectivity profile of the RSV ΔNS2/Δ1313/I1314L (Sanofi) candidate.

No safety concerns were identified after 1- and 2-dose administrations of either dose level of the investigational RSV ΔNS2/Δ1313/I1314L (Sanofi) vaccine or by baseline serostatus.

The vaccine virus shedding, and immunogenicity conclusions based on the IgA serostatus at baseline show marked vaccine take demonstrated at both dose levels, and 70% of vaccine RSV-naïve recipients attained a 4-fold response in serum neutralizing antibody responses post the second vaccine administration for both dose levels in RSV-naïve participants.

### REFERENCE NUMERAL KEY

10 medicament delivery system
11 medicament container/syringe
12 plunger rod
13 finger flange
14 outer side wall of container
15 medicament chamber
16 outlet
17 atomiser
18 piston
19 medicament
20 end surface of plunger rod
21 guide element/dose divider
22 body of guide element
23 aperture through guide element
24 guide slot
25 first section of guide slot
26 transition section of guide slot
27 second section of guide slot
28 biasing member
29 central aperture through biasing member
30 guide pins
31 projections on guide element
32 retaining elements
33 second transition section of guide slot
34 third section of guide slot
40 apparatus comprises finger flange 13, biasing member, guide element 21 and plunger rod 12
41 syringe barrel
42 syringe lip
43 projections of finger flange
P proximal end
D distal end

1. A medicament delivery system (10) comprising:
a syringe (11) comprising a chamber (15) for containing medicament and an outlet (16) at a distal end of the syringe through which medicament (19) can be expelled from the chamber;
a plunger rod (12) having a longitudinal axis, mounted within the syringe and slidable relative to the syringe in an axial direction of the plunger rod to cause medicament to be expelled from the chamber when medicament is within the chamber;
a guide element (21) at a proximal end of the syringe and comprising a central aperture (23) through which the plunger rod extends, wherein the guide element and the plunger rod are relatively rotatable about the longitudinal axis of the plunger rod between a blocking position and a release position; and
a cooperating guide mechanism provided on the plunger rod and the guide element (21);
wherein the guide mechanism permits the plunger rod to move in an axial direction over a first range of motion relative to the syringe from an initial position to an intermediate position to expel a first dose of medicament, and blocks the plunger rod from moving over a subsequent range of motion relative to the syringe to expel further medicament; and
wherein the plunger rod is free to be moved in an axial direction over the subsequent range of motion relative to the syringe to expel further medicament upon relative rotation of the guide element and the plunger rod from the blocking position to the release position.

2. A medicament delivery system (10) according to claim 1, wherein the guide element (21) is in a start position relative to the plunger rod (12) when the plunger rod is in the initial position, the guide element is in the blocking position relative to the plunger rod when the plunger rod is in the intermediate position, and the guide element is in the release position relative to the plunger rod when the plunger rod is free to be moved over the subsequent range of motion to expel further medicament.

3. A medicament delivery system (10) according to claim 2, wherein the start position and the intermediate position of the guide element (21) are rotationally offset about the longitudinal axis of the plunger rod (12).

4. A medicament delivery system (10) according to any one of claims 1 to 3, wherein the guide mechanism is configured to cause relative rotation between the guide element (21) and the plunger rod (12) as the plunger rod moves from the initial position to the intermediate position.

5. A medicament delivery system (10) according to any one of claims 1 to 4, wherein the guide mechanism comprises a follower (30) formed on one of the guide element (21) and the plunger rod (12), and a track (24) within which the follower is received, formed on the other of the guide element and the plunger rod.

6. A medicament delivery system (10) according to claim 5, wherein the track (24) comprises:
a first section (25) along which the follower (30) travels when the plunger rod (12) moves over the first range of motion,
a second section (27) along which the follower travels when the plunger rod moves over the subsequent range of motion, and
a transition section (26) connecting the first and second sections, and
wherein the plunger rod is prevented from moving over the subsequent range of motion until the follower has travelled along the transition section from the first section to the second section.

7. A medicament delivery system (10) according to claim 6, wherein the first section (25) is disposed at angle α relative to the longitudinal axis of the plunger rod (12), wherein the angle α is greater than 0° and less than 90°.

8. A medicament delivery system (10) according to any one of claims 1 to 7, wherein the guide element (21) is connected to the syringe (11) by a biasing member (28) configured to rotationally bias the guide element relative to the plunger rod (12) from the blocking position to the release position, and optionally wherein the biasing member (28) is resiliently deformable as the plunger rod (12) moves from the initial position to the intermediate position.

9. A medicament delivery system (10) according to claim 8 when dependent on claim 6 or claim 7, wherein the guide element (21) is free to move under an action of the biasing member (28) to the release position when the follower (30) enters the transition section (26) of the track (24) 10. A medicament delivery system (10) according to any one of the preceding claims, comprising a finger flange (13) at the proximal end of the syringe (11) opposite the outlet (16), and wherein the guide element (21) is coupled to the finger flange, and optionally when dependent on claim 8 or claim 9, wherein the biasing member (28) is secured to the finger flange (13).

11. A medicament delivery system (10) according to any one of the preceding claims, wherein the plunger rod (12) and the guide element (21) are configured such that substantially half of the medicament (19) in the chamber (15) is expelled as the plunger rod moves over the first range of motion, and substantially half of the medicament in the chamber is expelled as the plunger rod moves over the subsequent range of motion.

12. A medicament delivery system (10) according to any one of the preceding claims, further comprising an atomiser (17) at the outlet (16) of the syringe to facilitate insertion of the atomiser and the outlet into an orifice of a patient into which the medicament is to be delivered.

13. A medicament delivery system (10) according to any one of the preceding claims, wherein medicament (19) is received within the chamber (15) of the syringe (11).

14. A system (40) comprising:
a finger flange (13),
a guide element (21),
a plunger rod (12) for use with a syringe (11), the plunger rod receivable within an aperture (23) in the guide element, wherein the guide element and plunger rod are relatively rotatable about a longitudinal axis of the plunger rod between a blocking position and a release position, and
a cooperating guide mechanism provided on the plunger rod and the guide element,
wherein the system is attachable to the syringe such that the plunger rod is slidable within and relative to the syringe, and such that the guide mechanism permits the plunger rod to move over a first range of motion relative to the syringe to expel a first dose of medicament, and blocks the plunger rod from moving over a subsequent range of motion relative to the syringe to expel a further dose of medicament.

15. A system (40) according to claim 14, wherein the finger flange (13) is attachable to the syringe (11).

16. A method of operating a medicament delivery system (10) comprising a syringe (11) comprising a chamber (15) for containing medicament (16) and an outlet (16) at a distal end of the syringe, a plunger rod (12) having a longitudinal axis, mounted within the syringe, and slidable relative to the syringe to cause medicament (19) to be expelled from the chamber when medicament is within the chamber; a guide element (21) at a proximal end of the syringe and comprising an aperture through which the plunger rod extends; and a cooperating guide mechanism provided on the plunger rod and the guide element (21), the method comprising:
moving the plunger rod over a first range of motion relative to the syringe from an initial position to an intermediate position to expel a first dose of medicament;
the guide element blocking the plunger rod from moving over a subsequent range of motion relative to the syringe;
relatively rotating the guide element and the plunger rod from a blocking position to a release position;
moving the plunger rod over the subsequent range of motion relative to the syringe to expel a further dose of medicament.

17. A method of operating a medicament delivery system (10) comprising:
moving a plunger rod (12) over a first range of motion relative to a syringe (11) from an initial position to an intermediate position to expel a first dose of medicament;
blocking the plunger rod, via a guide element (21), from moving over a subsequent range of motion relative to the syringe;
relatively rotating the guide element and the plunger rod from a blocking position to a release position;
moving the plunger rod over the subsequent range of motion relative to the syringe to expel a further dose of medicament.
